(19) United States
(12) Patent Application Publication
(10) Pub. No.: US 2021/0339297 A1
(43) Pub. Date: Nov. 4, 2021

(54) SURGICAL INSTRUMENT CLEANING BRUSH ASSEMBLY FOR USE WITH A BORESCOPE

(71) Applicant: Kevin L. Stephenson, Coon Rapids, MN (US)

(72) Inventor: Kevin L. Stephenson, Coon Rapids, MN (US)

(21) Appl. No.: 17/240,042

(22) Filed: Apr. 26, 2021

Related U.S. Application Data
(60) Provisional application No. 63/017,349, filed on Apr. 29, 2020.

(51) Int. Cl. B08B 9/04; A61B 90/70; A46B 17/02

(52) U.S. Cl. CPC B08B 9/04; A61B 90/70; B08B 2209/04; A61B 2090/701; A46B 2200/3013; A46B 17/02

(57) ABSTRACT

A brush assembly for use with a borescope in cleaning a surgical instrument lumen. The brush assembly includes a shaft, a plurality of bristles or other cleaning materials, and at least one retention clip. The bristles or other cleaning materials extend from the shaft or other cleaning material. The retention clip is assembled to the shaft proximal the bristles. In some embodiments, the retention clip is permanently attached to the shaft. Regardless, the retention clip is configured for selective mounting to a borescope guide tube. Upon mounting to the borescope guide tube, the brush assembly can be deployed to clean a surgical instrument lumen, with the borescope providing a user with visual confirmation of cleaning effective by the bristles.

### CROSS-REFERENCE TO RELATED APPLICATIONS

This Non-Provisional Patent Application claims the benefit of the filing date of U.S. Provisional Patent Application Ser. No. 63/017,349, filed Apr. 29, 2020, the entire teachings of which are incorporated herein by reference.

### BACKGROUND

The present disclosure relates to the simultaneous cleaning and inspection of surgical instrument lumen(s). More particularly, it relates to systems for inspection systems appropriate for use with the cleaning of a surgical instrument lumen, such as an endoscope lumen or channel, in a liquid (e.g., water) environment.

A plethora of different surgical instruments incorporate a tubular design in which one or more lumens or channels are utilized to facilitate caregiver interface with a target site inside the patient's body. For example, irrigation, suction, additional instrument(s), etc., are commonly delivered to an internal treatment site via the lumen(s) of a surgical instrument. Endoscopes are but one example of a surgical instrument providing at least one lumen (sometimes referred to as a “channel” in the context of endoscopes).

Regardless of exact form, under circumstances where the particular surgical instrument is intended and designed for repeated use, the instrument must be sterilized or high level disinfected prior to each use. And prior to the sterilization or high level disinfection, all debris and foreign matter must be removed from all surfaces of the instrument, both inside and out. In other words, the internal surfaces of the surgical instrument otherwise defining the instrument's lumen(s) must be cleaned.

Currently, the process after a procedure to clean a surgical instrument starts in the operating field. A surgical technician wipes the outside of the surgical instrument and flushes out the lumen(s) with sterile water or enzymatic solution (depending upon the procedure for which the surgical instrument was used). The next step is to manually clean the surgical instrument with an enzymatic solution in a decontamination or reprocessing area of the caregiver's facility. This process is performed at a sink filled with enzymatic solution. To clean surgical instrument lumen(s) (e.g., the lumen(s) or channel(s) of an endoscope), a technician inserts a brush into the lumen while submersed in the enzymatic solution. The technician manipulates the brush with back-and-forth and twisting motions, scrubbing debris and residue from the internal surfaces. With sufficient time and effort, this traditional cleaning technique can be effective in removing all debris from the lumen surfaces. However, the technician has no way of knowing in advance the time and effort required for a particular cleaning task as the physical constraints and type(s) of debris encountered vary widely. Moreover, because the lumen surfaces are internal or “hidden” relative to an exterior of an otherwise non-transparent surgical instrument (e.g., typically formed of surgical grade stainless steel or the like), the technician has no way of visually evaluating cleanliness of the lumen surfaces with the naked eye. It is exceedingly difficult to fully clean what the technician cannot see. An additional concern with traditional brush cleaning is that bristles of the brush may detach from the cleaning tool and problematically become lodged within the lumen. To confirm that no brush particulates or foreign debris are left in the lumen, many institutions have the technician, after using the brush to clean the lumen, use a borescope to visually confirm that the internal areas of the device are cleaned to expectations. Today, this two-step process of cleaning and inspection can be performed at the sink with waterproof borescopes

Multiple surveys and clinical evaluations have documented that surgical instrument lumens (such as endoscopic channels) are still not fully clean following traditional cleaning. Most have reported that 60%-80% of lumens are still dirty when inspected following a traditional cleaning process.

### SUMMARY

The inventor of the present disclosure recognized that a need exists for devices and methods that address one or more of the above problems.

Some aspects of the present disclosure are directed toward a surgical instrument cleaning brush assembly and corresponding methods of use. The brush assembly includes a surgical instrument cleaning brush attached to (e.g., permanently attached to) at least one retention clip. The retention clip is configured for releasable assembly to an exterior surface of a borescope. During use, the retention clip is fastened to a borescope; the brush assembly/borescope is then inserted into the surgical instrument lumen to be cleaned. The retention clip holds the cleaning brush in place relative to the borescope, affording the technician the ability to clean the lumen or channel with the cleaning brush (e.g., the borescope is moved in a back-and-forth manner, with this movement being transferred onto the cleaning brush) while visually observing the cleaning action at the time of cleaning. Direct visualization during the cleaning process allows the technician to visually confirm they have cleaned the lumen(s) to their expectations, thus increasing efficiency and efficacy in the cleaning process. At the conclusion of a particular cleaning task, the brush assembly can be disassembled from the borescope and then disposed of or refurbished/cleaned for subsequent re-use with another cleaning task.

### BRIEF DESCRIPTION OF THE DRAWINGS

FIG. 1 is a perspective view of a portion of a surgical instrument cleaning brush assembly in accordance with principles of the present disclosure;

FIG. 2A is a perspective view of a clip useful with the surgical instrument cleaning brush assembly of FIG. 1;

FIG. 2B is an end view of the clip of FIG. 2A;

FIG. 3 is a side view illustrating the surgical instrument cleaning brush assembly of FIG. 1 mounted to a borescope;

FIG. 4 is an enlarged, perspective view of a portion of the arrangement of FIG. 3;

FIG. 5 illustrates use of the assembly of FIG. 3 in cleaning a lumen of a surgical instrument; and

FIG. 6 is an image of a cleaning device lumen being cleaned by the assembly of FIG. 3.

### DETAILED DESCRIPTION

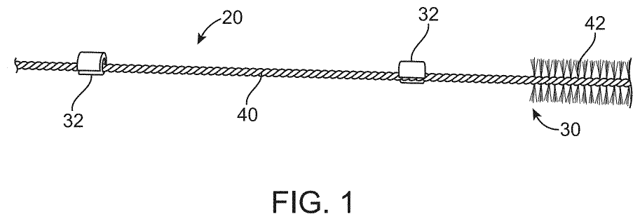

One embodiment of a surgical instrument cleaning brush assembly 20 in accordance with principles of the present disclosure in shown in FIG. 1. The brush assembly 20 includes a brush 30 and at least one retention clip 32. Details on the various components are provided below. In general terms, the retention clip(s) 32 is attached to the brush 30 and is configured for selective securement to a borescope (not shown). When mounted to a borescope, the borescope/brush assembly 20 can be utilized to perform a surgical instrument lumen cleaning process in which the borescope provides direct visualization of the cleaning being effected by the brush assembly 20.

The brush 30 can assumes various forms appropriate for cleaning a surgical instrument lumen or channel, and in some embodiments is, or is akin to, a brush conventionally used for manually cleaning of a surgical instrument lumen or channel. The brush 30 generally includes a shaft 40 and carrying or secured to a plurality of bristles 42. The shaft 40 can have various forms, and can be generally constructed to exhibit radial flexibility and axial rigidity. For example, in some non-limiting embodiments, the shaft can be length of twisted metal wire(s). Other constructions are also acceptable. While the shaft 40 is generally shown as being a single, continuous body, in other embodiments the shaft 40 can be defined or generated by two or more structures attached to one another.

The bristles 42 are each attached or otherwise secured to the shaft 40 so as to extend in a generally radial fashion relative to a central axis of the shaft 40. A bias or pattern can be generated by an arrangement of the bristles 42 along the shaft 40. The bristles 42 can assume various forms, and in some embodiments, are or include a polymer material. In some non-limiting examples, the bristles 42 are nylon, polypropylene, microfiber, etc., although other materials are also acceptable including materials developed in the future that are beneficial for cleaning surgical instrument lumens. The bristles 42 can have various diameters, for example in the range of 1-25 mm. A length of each of the bristles 42 (e.g., distance of radial extension from the shaft 40) can vary as a function of an intended end-use cleaning application (e.g., a size of the surgical instrument lumen to be cleaned), and in some embodiments can be in the range of 5-25 mm. Regardless, an outer diameter collectively defined by the bristles 42 is greater than an outer diameter of the shaft 40.

The shaft 40 defines an overall length of the brush 30, with the bristles 42 being secured to or extending along a distal region of the shaft 40. A length of the shaft 40, and thus a length of the brush 30, can vary as a function of an intended end-use application. In some embodiments, the shaft 40 can have a length in the range of 10-318 cm (4-125 inches). Regardless, at least some, optional all, of the shaft 40 proximal the bristles 42 can be exposed or otherwise available for securement to the retention clip(s) 32.

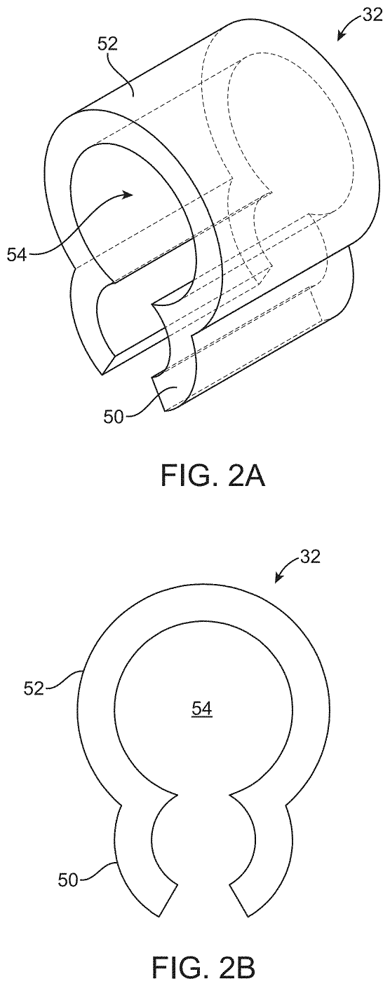

The retention clip(s) 32 can assume various forms conducive to selective connection or mounting to a borescope. With additional reference to FIGS. 2A and 2B, the retention clip 32 can include or define a base section 50 and a connection section 52. The base section 50 is configured for attachment, optionally permanent attachment, to the brush shaft 40. In some embodiments, the retention clip 32 can be adhered, bonded, welded, molded, etc. to or about the shaft 40. In other embodiments, the base section 50 can be configured for selective attachment to the shaft 40 (e.g., the base section 50 can define an open-ended aperture sized to receive and frictionally retain the shaft 40). The connection section 52 extends from the base section 50 and is configured to define a channel 54 sized and shaped for selective or releasable mounting to an exterior of a conventional borescope (not shown). In general terms, the channel 54 is open at both the distal and proximal ends of the retention clip 32. In some embodiments, the connection section 52 can be or can be akin to a closed circle or loop whereby the channel 54 is accessible only at the distal and proximal openings. In other embodiments, the connection section 52 can be or can be akin to a split circle (e.g., a passage is formed through a thickness of the connection section 52 opposite the base section 50 and through which a body (e.g., a borescope) can be inserted into/removed from the channel 54).

The retention clip(s) 32 can be formed from a material that will not readily degrade in the presence of cleaning liquids or solutions commonly employed for surgical instrument cleaning tasks. A material of the retention clip(s) 32 is optionally selected to exhibit a relatively high coefficient of friction with an expected material and/or surface features of a conventional borescope so as to more readily establish a non-sliding interface when assembled thereto. In other optional embodiments, at least an interior surface of the connection section 52 can include or form surface features that provide enhanced purchase with the surface of a conventional borescope.

As reflected by FIG. 1, in some embodiments, the brush assembly 20 includes two of the retention clips 32 longitudinally spaced from one another along a length of the shaft 40. Alternatively, more or less of the retention clips 32 (including a single retention clip 32) can be provided. Regardless, the distal-most retention clip 32 is arranged along the shaft 40 so as to be longitudinally spaced from a proximal-most one of the bristles 42. For example, in some embodiments, where the distal-most retention clip 32 is permanently assembled to the shaft, a longitudinal spacing on the order of 1-15 cm is established between the distal-most retention clip 32 and proximal-most bristle 42. With embodiments in which two or more of the retentions clips 32 are provided and are permanently assembled or attached to the shaft, the retention clips 32 are arranged such that the corresponding channels 54 are substantially axially aligned (i.e., within 10% of a truly axially aligned relationship).

As implicated above, the brush assembly 20 is configured for use with a borescope. The borescopes with which the present disclosure can be useful can assume various forms as known in the art, and borescopes of the present disclosure generally include a guide tube (or “borescope guide tube”) carrying an optical relay system by which an image at an objective end of the guide tube is delivered or relayed to an imaging end. Further, the guide tube incorporates or carries various optical components (e.g., fiber optics, light guides, etc.) for delivering light to the objective end (and illuminating the area to be observed at the objective end). The guide tube can have a relatively small outer diameter, for example on the order of 2 mm in some non-limiting embodiments. A light port is provided with the borescopes of the present disclosure, adapted to interface with the illumination source in directing light from the illumination source into and along the guide tube (that in turn is configured to, or carries components configured to, transmit or direct the light to the objective end). The guide tube can have a flexible construction as is known in the art.

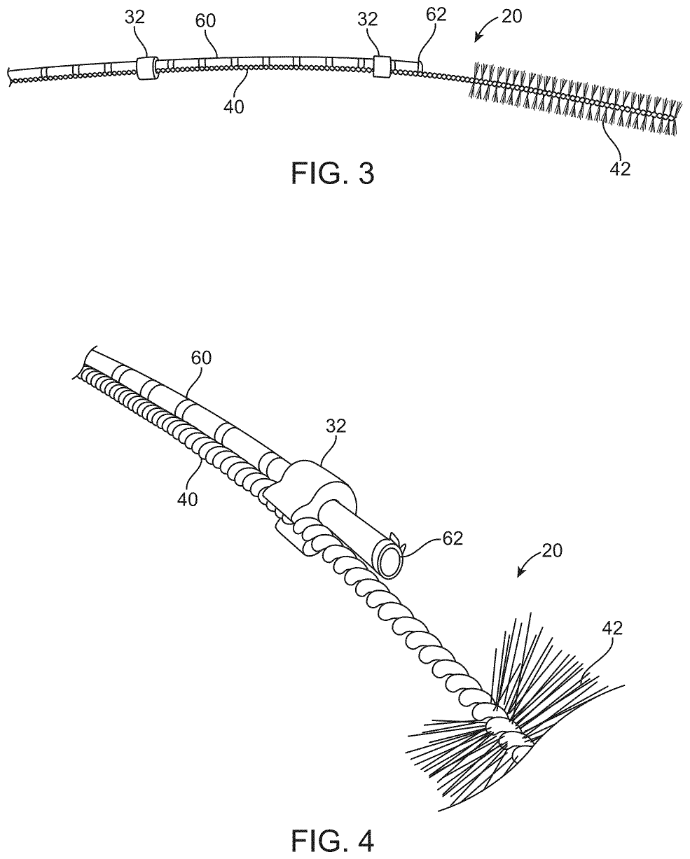

With the above in mind, FIGS. 3 and 4 depict the brush assembly 20 mounted to the exterior of a borescope guide tube 60. The borescope guide tube 60 is received within the channel 54 (FIG. 2) of each retention clips 32, locating the bristles 42 to be slightly distal an objective end (or distal end) 62 of the borescope guide tube 60. For example, in some non-limiting embodiments, the brush assembly 20 can be connected to the borescope guide tube 60 by inserting the distal end 62 into and then through the channel 54 of the retention clip(s) 32. Other selective mounting configurations or techniques are also acceptable. Regardless, a user can select a desired location of the bristles 42 relative to the objective end 62.

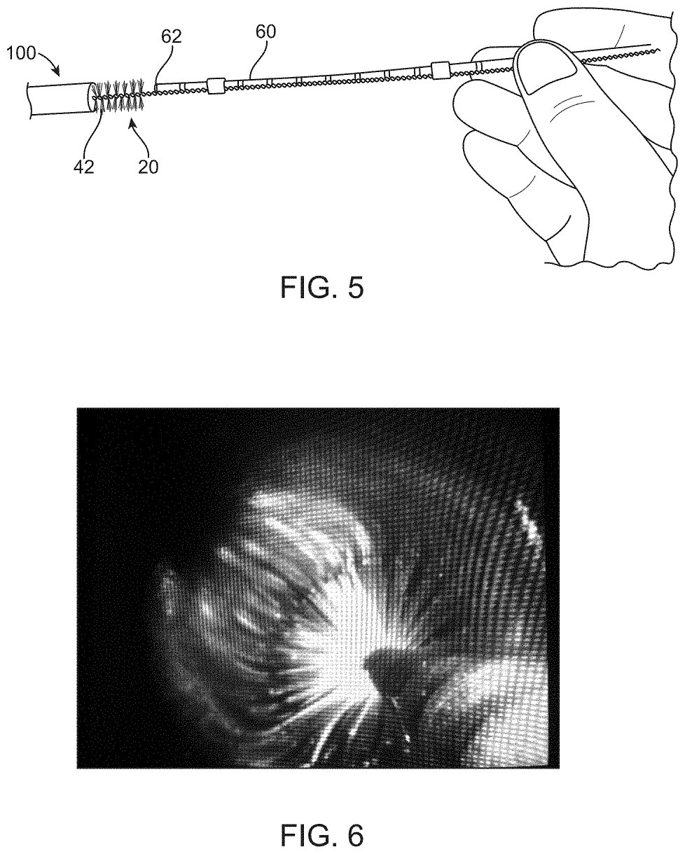

Upon final assembly, the shaft 40 is maintained side-by-side with the borescope guide tube 60. Relative to the segment of the borescope guide tube 60 along which the shaft 40 is located, the shaft 40 extends substantially parallel (e.g., within 5% of a truly parallel relationship) to the borescope guide tube 60, and can flex or deflect in tandem with the corresponding segment of the borescope guide tube 60. During use, then, and as generally reflected by FIG. 5, the combination borescope/brush assembly 20 can be used to access, visualize and clean a lumen or channel of a surgical instrument 100, with the bristles 42 inserted into the lumen followed by the objective end 62. The borescope guide tube 60 can then be moved back-and-forth within the surgical instrument lumen; because the brush assembly 20 is secured to the borescope guide tube 60 by the retention clip(s) 32, movement of the borescope guide tube 60 is transferred to the shaft 40 and thus the bristles 42. The shaft 40 is axially rigid, maintaining the bristles 42 at the selected location relative to the object end 62. As a result, the bristles 42 effect cleaning of the surgical instrument lumen with back-and-forth movement of the borescope guide tube 60, with the borescope affording the technician the ability to view the surgical instrument lumen throughout the cleaning process. FIG. 6 is an image of the brush assembly in a surgical instrument lumen being observed with the borescope. Upon completion of the cleaning process, the brush assembly 20 can be removed from the borescope guide tube 60 and disposed of, or subjected to appropriate cleaning processes that render the brush assembly 20 available for re-use.

Although the present disclosure has been described with reference to preferred embodiments, workers skilled in the art will recognize that changes can be made in form and detail without departing from the spirit and scope of the present disclosure.

What is claimed is:

1. A brush assembly for use with a borescope to clean a surgical instrument lumen, the brush assembly comprising:
a shaft;
a plurality of bristles extending from the shaft; and
at least one retention clip secured to the shaft proximal the bristles and configured for selective mounting to a borescope guide tube.

2. The brush assembly of claim 1, wherein the retention clip is permanently attached to the shaft.

3. The brush assembly of claim 1, wherein the retention clip defines a base section secured to the shaft and a connection section extending from the base section, and further wherein the connection section is configured to define a channel sized for selective or releasable mounting to an exterior of a borescope guide tube.

4. The brush assembly of claim 3, wherein the retention clip defines a proximal end opposite a distal end, and further wherein the channel 54 is open at both the distal and proximal ends.

5. The brush assembly of claim 4, wherein the connection section forms a closed circle such that the channel is accessible only at the distal and proximal ends.

6. The brush assembly of claim 4, wherein the connection section defines a split circle generating a passage opposite the base section and through which a borescope guide tube can be inserted into the channel.

7. The brush assembly of claim 1, wherein the brush assembly includes at least two of the retention clips.

8. A method of cleaning a lumen of a surgical instrument, the method comprising:
receiving a brush assembly including a shaft, a plurality of bristles extending from the shaft, and at least one retention clip secured to the shaft proximal the bristles;
mounting the retention clip to a guide tube of a borescope; and
inserting the combination borescope guide tube and bristles into a lumen of a surgical instrument.

9. The method of claim 8, wherein the step of inserting includes manipulating the borescope guide tube to cause the bristles to clean an interior surface of the surgical instrument otherwise defining the lumen.

10. The method of claim 9, wherein the step of inserting further includes inspecting the interior surface via the borescope.

11. The method of claim 10, further comprising:
removing the brush assembly from the borescope guide tube.
